(19) United States
(12) Patent Application Publication
(10) Pub. No.: US 2010/0258132 A1
(43) Pub. Date: Oct. 14, 2010

(54) Headgear pad for CPAP interface

(76) Inventor: Karen Leigh Moore, Stanardsville, VA (US)

Correspondence Address: KAREN LEIGH MOORE, 157 SUNSET DRIVE, STANARDSVILLE, VA, 22973, US

(21) Appl. No.: 11/522,156

(22) Filed: Sep. 16, 2006

Related U.S. Application Data
(60) Provisional application No. 60/719,320, filed on Sep. 22, 2005.

(51) Int. Cl. A61M 16/06

(52) U.S. Cl. 128/207.11

(57) ABSTRACT

The invention is a method for use and a strap pad for CPAP interfaces to prevent skin irritation, discomfort, indentations, and allergies. The invention incorporates a generally rectangular elongated strip of flexible material having sufficient pile to provide padding. The material is fastened into a hollow tube for surrounding headgear straps. The flexible material incorporates an outer layer of micro fleece or similar material whereby protecting the skin, the softness mentally calms against the face when falling to sleep.

The protective pad also incorporates a plurality of pairs of attachments extending from the opposite ends which facilitate the placement of the pad along the straps. Further, the invention provides color and design for an otherwise unattractive facial apparatus. Persons suffering from obstructive sleep apnea are often assigned this apparatus for therapy. The decorative quality improves the appearance and the attitude of the wearer thereby facilitating adjustment to a challenging change in lifestyle and thus improves compliance of therapy for this serious condition.

### CROSS-REFERENCE TO RELATED APPLICATIONS

This application claims the priority, under 35 U.S.C. Sec. 119, of the U.S. Provisional Patent Application Ser. No. 60/719,320 filed Sep. 22, 2005 by the present inventor. The entire disclosures of this prior application are hereby incorporated by reference in its entirety.

### STATEMENT REGARDING FEDERALLY SPONSORED RESEARCH OR DEVELOPMENT

Not Applicable

### STATEMENT REGARDING SEQUENCE LISTING OR PROGRAM

Not Applicable

### FIELD OF THE INVENTION

This invention relates generally to the field of respiration, or breathing and/or ventilation masks. In particular, the invention relates to the headgear supports of masks and nasal pillows interfaces used in the treatment of sleep apnea.

### BACKGROUND

### Prior Art

Obstructive sleep apnea syndrome (commonly referred to as obstructive sleep apnea, OSA, and/or sleep apnea) is a medical condition that has as one symptom repeated prolonged episodes of cessation of breathing throughout the night. This is generally a condition in which a person's airways becomes blocked or restricted due to the over-relaxation of the muscles and tissues in the throat during sleep.

A restrictive air supply impairs the oxygen flow to the lungs and, in turn, to the brain and other areas of the body, causing O₂ de-saturations.

An individual with OSA can stop breathing, or suffer an apnea event/episode, many times (typically from 10 to 30 times an hour) each night. The apneas are generally followed by sudden reflexive attempts to breathe. As a result, an individual suffering from OSA may experience fragmented sleep and, in certain cases, total deprivation of sleep stages III, IV and REM sleep. Such non-restorative sleep results in daytime drowsiness, headaches, weight gain, limited attention span, memory loss, lethargy, inability to maintain concentration and/or depression. OSA has also been linked to increased hypertension and greater risk of stroke, heart disease, and diabetes if left untreated. Untreated sleep apnea is also a leading cause of automobile accidents.

Effective treatment requires the airways to remain open during sleep. Modern respiratory therapy utilizes a “continuous positive airway pressure” (CPAP) machine and a ventilation interface during sleep in order to keep the airways open. A variety of CPAP machines can provide this treatment. BiPAP and VPAP are two specialized examples of CPAP machines that vary the pressure received based on the patient's breathing patterns. All positive airway pressure machines use an interface to deliver ambient air, under pressure, to the patient's airways which effectively forms a stent for the trachea. This interface usually consists of a nasal mask, full face mask (covering both the nose and mouth), or nasal pillows attached to the patient's face with straps or headgear, and connected to the CPAP machine by way of an air hose.

No matter what type of OSA interface is used, holding it in place over the nose and/or mouth can be difficult due to the air pressure blowing into it. Some interfaces employ an arrangement of straps which encircle the head of the user. Such straps or headgear are uncomfortable and rough on the edges (particularly when the Velcro adjustment tab extends to the edge of the strap). The headgear can cause skin irritations and allergic reactions. The straps of the headgear, secured sufficiently to prevent mask leaks, may cause indentations on the face that can remain hours after removal of the mask.

Another difficulty results in psychological aspects of the treatment. The CPAP machine and mask must be used any time the person sleeps, including naps, for the duration of the patient's life in order to be effective against the long-lasting dangers of OSA. The mask and headgear are unnatural, cumbersome, medical-looking, uncomfortable, as well as unattractive on the face. CPAP treatment has even been the cause of personal relationships being destroyed. The hospital look of the mask during use and the marks remaining on the face after use are a continual reminder of the patient's medical condition.

As a result of the varieties of difficulties and discomforts, some patients remove their masks in the night, at times not having realized that they have done so. Some patients choose to discontinue treatment for OSA because of the many difficulties associated with adjusting to the CPAP masks and nightly treatment. Without treatment compliance, OSA progressively becomes more severe as a person ages.

The mask headgear, usually comprised of a kind of neoprene straps, requires regular cleaning because of the body oil and sweat accumulation with use. Masks and headgear also wear out from repeated nightly use and need to be replaced as often as every 3 months. Considering the cost of these devices, replacement gets expensive when considering the lifetime need of the patient.

Durable Medical Equipment suppliers (DMEs) have limited style choices available in masks and CPAP machines for the patients. Most of the masks and headgear are very similar in appearance and structure. Most patients have little knowledge about OSA when they are diagnosed and know very little about the treatment options. The patients are further handicapped due to the very nature of the disorder, and cannot think as clearly as after receiving proper treatment, so are often given the mask and machine the DME selects for them. Having little input in the choice of equipment can negatively affect the dedication the patient has toward the treatment, especially considering the variety of challenges it presents and the time it can require to feel the benefits of the therapy.

The invention of a colorful, soft headgear strap pad alleviates many of the problems associated with OSA therapy listed above and helps patients remain compliant with their treatment. Compliant treatment allows the patients to experience a much greater quality of life.

In the related art, numerous attempts at treatment of OSA have been made. These attempts have included new masks and headgear designs; some have included designs to reduce the amount of strap area against the skin. A search of the prior art did not disclose any patents that read directly on the claims of the invention herein disclosed; however, the following patents were considered relevant:

| U.S. Pat. No. | Inventor | Issue Date |
|---|---|---|
| 3,817,245 | Mildred Kroeger | Jun. 18, 1974 |
| 4,472,838 | Susan Pompa | Sep. 25, 1984 |
| 4,575,874 | William M. Johnson | Mar. 18, 1986 |
| 4,945,576 | Ardella R. Melton | Aug. 7, 1990 |
| 5,019,064 | Kasriel Eilender | May 28, 1991 |
| 5,048,542 | J. Kent Murray | Sep. 17, 1991 |
| 5,509,409 | Marjorie F. Weatherholt | Apr. 23, 1996 |
| 5,687,743 | Isabell Goodwin | Nov. 18, 1997 |
| 5,893,365 | Clarence D. Anderson | Apr. 13, 1999 |
| 5,921,239 | Shawn R. McCall et al. | Jul. 13, 1999 |
| 6,012,455 | Joseph Goldstein | Jan. 11, 2000 |
| 6,019,101 | Ronald L. Cotner et al | Feb. 1, 2000 |
| 6,026,811 | Romaine A. Settle | Feb. 22, 2000 |
| 6,196,223 B1 | William A. Belfer et al. | Mar. 6, 2001 |
| 6,381,760 B1 | John K Lampe et al. | May 7, 2002 |
| 6,386,198 B1 | Florence Rugless | May 14, 2002 |
| 6,412,488 B1 | Shari S. Barnett et al. | Jul. 2, 2002 |
| 6,422,238 B1 | Perry D. Lithgow | Jul. 23, 2002 |
| 6,470,886 B1 | Bernadette Jestrabek-Hart | Oct. 29, 2002 |
| 6,619,288 B2 | Jason A. Demers et al. | Sep. 16, 2003 |
| 6,634,358 B2 | Philip Rodney Kwok | Oct. 21, 2003 |
| 6,886,564 B2 | Colin E. Sullivan et al. | May 3, 2005 |
| 6,918,394 B2 | Narihiko Matsuda et al. | Jul. 19, 2005 |
| 6,926,004 B2 | Gerhard Schumacher | Aug. 9, 2005 |
| 6,938,620 B2 | Charles E. Payne, Jr. | Sep. 6, 2005 |
| 6,966,074 B2 | Moon Young Huh | Nov. 22, 2005 |
| 7,000,611 B2 | Walter G. Klemperer | Feb. 21, 2006 |
| 7,017,576 B2 | Gregory James Olsen et al. | Mar. 28, 2006 |
| 7,047,972 B2 | Anthony Michael Ging et al. | May 23, 2006 |
| 7,069,933 | Philip Rodney Kwok et al. | Jul. 4, 2006 |

### OBJECTS AND ADVANTAGES

While there are devices that provide treatment options for OSA, these do not disclose a successful padding solution for the headgear of CPAP interfaces. In this respect, the nasal headgear padding described herein departs from any present designs in providing comfort, physically and mentally, and style. Compliance improves dramatically when the patient has comfortable options for the treatment of OSA.

The present invention discloses a padding tube which covers the CPAP headgear strap contiguous to the face. This extremely soft pad protects the wearer's face and lower ear from any irritation caused by roughness inherent in the headgear strap itself or the Velcro tab, if it is exposed to the face. The present invention also protects the facial skin and ear from any allergic reaction to the materials of the headgear by forming a physical barrier. The material of the present invention is such that it promotes air circulation under the straps, controlling moisture build-up and discomfort. By rounding the edge of the strap, the present invention prevents lasting indentations from forming on the face caused by side sleeping and resting the cheek on the strap. The softness of the present invention is comparable to a very soft blanket against the cheek, adding physical as well as psychological comfort to the treatment of OSA. Soft comfortable straps increase the ease of falling asleep and staying asleep.

The present invention discloses that padding the straps and preventing facial indentations allow the wearer of the CPAP mask to only think about the treatment of OSA at night when going to bed. It allows the medical nature of the disorder to be a private matter instead of having to explain to others the following morning about the marks on their face that were left behind by the straps at night. Psychological benefits include a more positive self-image and improved social interaction. The present invention improves the outward medical appearance of the headgear by being colorful and very soft, as well as allowing personalization. It also allows individual style for each person by being available in many colors and material finishes. The present invention can match other clothing worn to bed and making it feel more like an accessory. The present invention can be changed often when the wearer wants a different style or color on the headgear, adding an element of fun to an otherwise unpleasant situation.

The present invention discloses that covering the headgear strap adjacent to the face and ear prevents soiling of the headgear and prevents wear of the headgear caused by rubbing against the bed sheets and pillowcase. The present invention is easily removed, washed, dried and reinstalled. The Velcro on the strap picks up less lint in the hook portion by being covered, thus extending the life of the attachment point of the strap to the mask. Prolonging the useful life of the headgear requires replacement less often and thereby saves the patient costs associated with treatment. When the pad wears out, it is much more economical to replace than the entire headgear, which some DMEs will only sell as a unit with a new mask.

The present invention discloses that by adding padding to the present headgear strap, a more secure attachment is made to the mask. By changing the angle in which the strap attaches to the mask, there is less pressure required of the straps to secure the mask to the airways and the mask forms a better seal with the face, allowing the CPAP machine to provide therapy more effectively.

Aside from the obvious comfort solutions that the padding invention herein offers, the telltale facial indentations due to overnight usage of CPAP masks and interfaces are eliminated. This helps to ensure the user's privacy by not allowing others to know they are undergoing treatment nightly, which could lead to greater compliance. Since the padding invention herein described can offer a more attractive and aesthetically-pleasing appearance, it can alleviate the embarrassment a user can feel from wearing their mask in front of a bed partner, once again, creating greater compliance.

This present headgear pad has experienced increasing sales every month for almost a year that it has been offered to the public. Need is there for this unique invention for continued treatment for obstructive sleep apnea.

Prior art treatment options for OSA do not adequately address a successful padding solution for the headgear of CPAP interfaces. The present invention described herein deviates from any prior art in providing improvements in the way of comfortable options for OSA patients and thereby improves compliance. Still further objects and advantages will become apparent from a consideration of the ensuing description and drawings.

### SUMMARY

In accordance with the present invention, the Headgear Pad for CPAP Interface comprises a resilient, sufficiently bulky fabric tube formed in a fashion to provide a secure padding for the headgear straps against the face of a sleep apnea patient. This invention increases compliance of therapy by making the treatment more comfortable, more attractive, as well as more private.

### DRAWINGS

### Figures

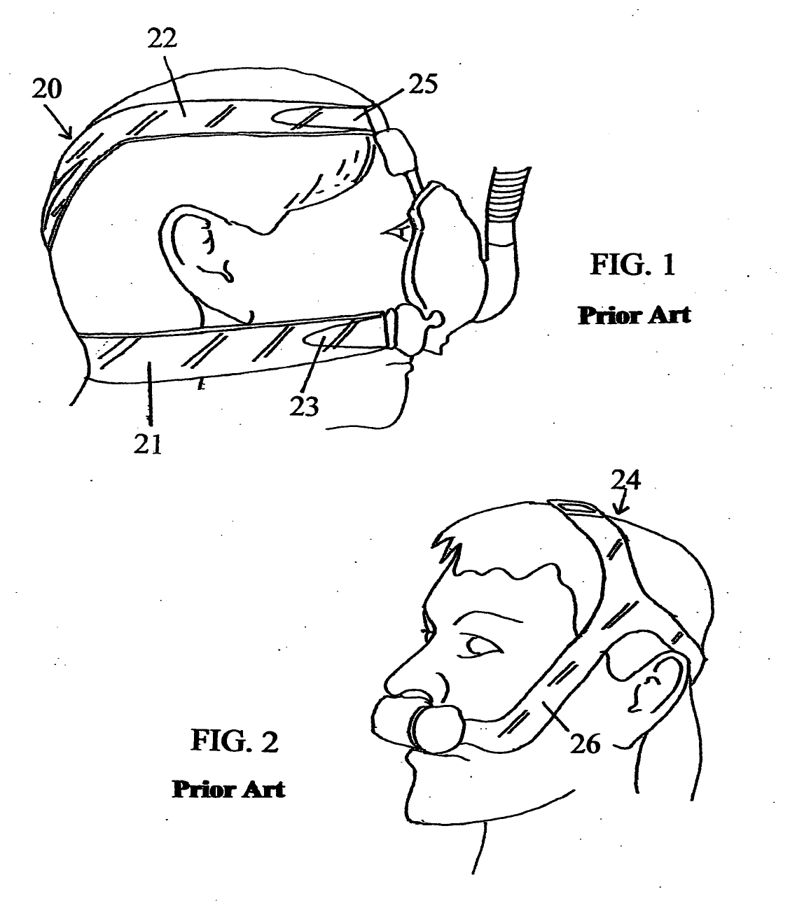

FIG. 1 shows prior art of a standard four strap headgear without the invention installed.

FIG. 2 shows prior art of a nasal pillow headgear without the invention.

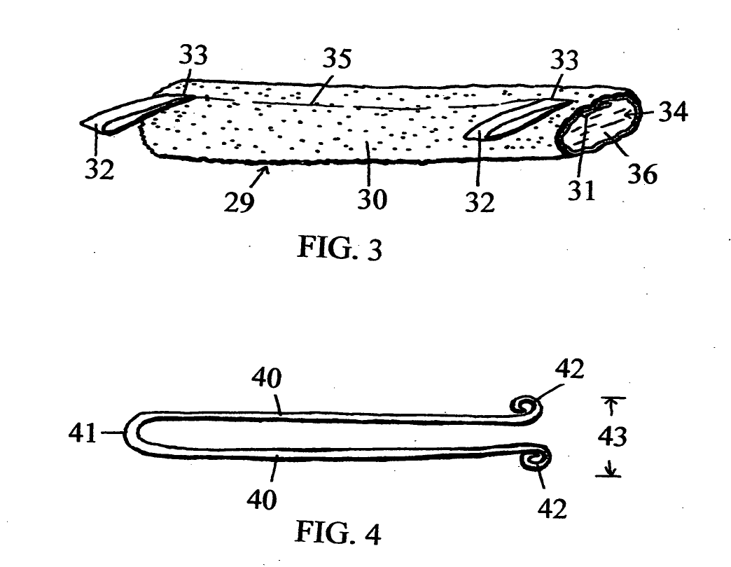

FIG. 3 shows the padding tube with the ribbon tabs.

FIG. 4 shows the wire installation tool for putting the tube on the strap.

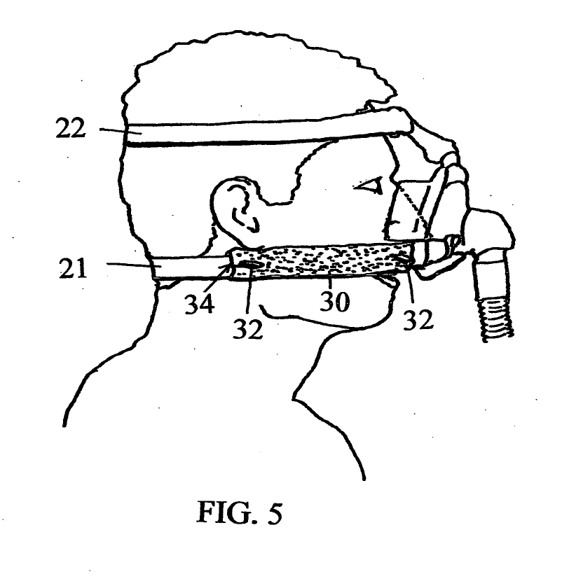

FIG. 5 shows the headgear pad invention installed on the lower strap of a standard headgear.

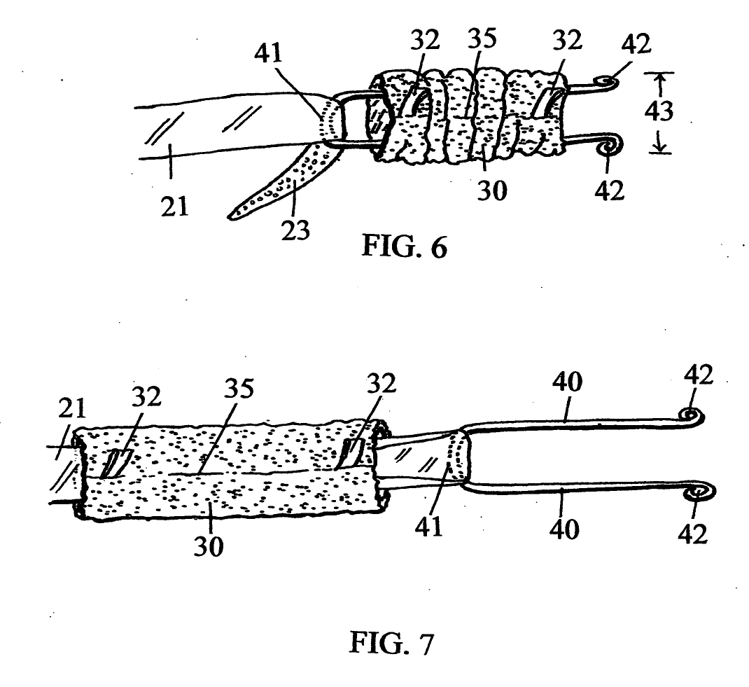

FIG. 6 shows the wire tool inserted in the padding tube and the strap from the headgear.

FIG. 7 shows the tool and the padding tube after the tube has been slid over the strap of the headgear.

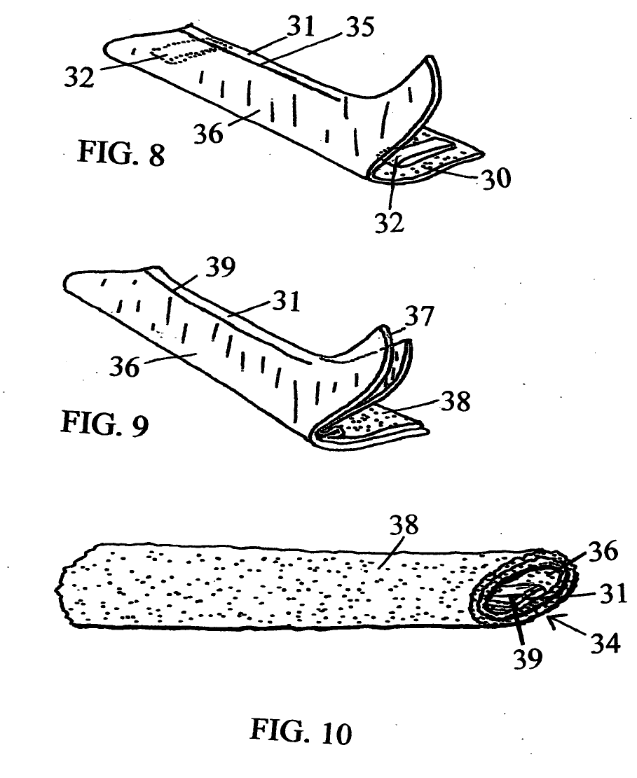

FIG. 8 shows the method of construction for the preferred embodiment of the invention.

FIG. 9 shows an alternative two-layer construction of the invention.

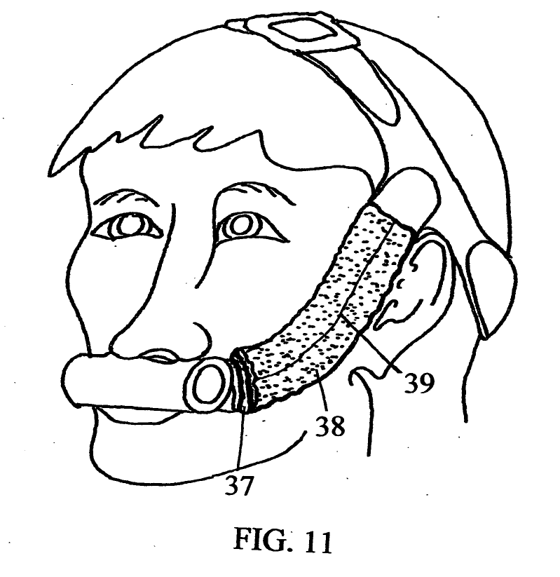

FIG. 10 shows an alternative double layered pad.

FIG. 11 shows an alternative double layered pad installed on a nasal pillow headgear.

### REFERENCE NUMERALS

20 Standard four strap headgear for CPAP face mask
21 Lower strap of standard headgear for CPAP face mask
22 Upper strap of standard headgear for CPAP face mask
23 Fastening tab of lower strap on standard headgear for CPAP face mask
24 Example headgear for CPAP nasal pillows interface
25 Fastening tab of upper strap on standard headgear for CPAP face mask
26 Cheek strap of example headgear for CPAP nasal pillows interface
29 Body of padding for CPAP four strap headgear
30 Outer surface of padding tube
31 Seam allowance of seam in padding tube
32 Attachments extending from seam in padding tube
33 Insertion point of the attachment in the seam
34 Hollow area extending through padding
35 Seam line of thread holding sides of tube together
36 Interior unexposed surface of padding tube
37 Inner layer of multi-layered padding tube
38 Outer layer of multi-layered padding tube
39 Seam line of thread holding multi-layers of padding tube
40 Insertion tool leg
41 U shaped bend in insertion tool
42 Curled ends of insertion tool
43 Distance from outside edge of one curled end to same on other curled end

### DETAILED DESCRIPTION OF DRAWINGS

FIGS. 1 and 2 show prior art examples of conventional CPAP facial mask headgear 20 and nasal pillows headgear 24. The straps 21, 22 of the standard four strap headgear 20 consist of elastic or non-elastic strap material with rough sides and Velcro tab 23 to attach to the mask. The side strap 26 on the example of the nasal pillows headgear 24 are made of a plastic semi-rigid material with no padding. These straps 21, 22 and side strap 26 are worn in a manner which pressure is applied to the head to hold the interfaces in proper alignment to deliver pressurized air to the wearer. The straps 21, 22 have no obvious method to prevent irritation or allergies.

FIG. 3 shows the present invention headgear pad to cover the straps 21, 22 on headgear in FIG. 1. In the preferred embodiment, the headgear pad FIG. 3 is constructed of one directional stretch material 29 that can stretch transversely of its length. While any one directional stretch material of sufficient thickness to pad the skin can be utilized, polyester micro fleece is preferred because of its soft outer surface 29 and tendency to remain free from raveling when cut, allowing less bulk at the terminal ends of the headgear pad FIG. 3. Colorful or decorative material may be used to further personalize the strap pad. Attachments 32 of contrasting or similar color of the material 29 extend from the attachment point 33 in the seam line 35. The seam line 35 of thread holds the opposing long sides of the material 29 including the attachments 32 securely into the shape of a tube with a hollow 34 being appropriately sized for the headgear strap 21 extending the length of the tube. Seam allowance 31 extending inwardly into the hollow 34 run the length of the tube. The unexposed side 36 of the one way stretch material 29 has texture significant to provide friction against the straps 21, 22 of the headgear 20 to prevent slippage.

FIG. 4 shows an installation tool for the headgear pad FIG. 3. In the preferred embodiment, this device consists of wire that has a U shape bend 41 forming a tool narrow enough to partially slide through the padding tube FIG. 3. The installation tool FIG. 4 has sufficient length of the leg 40 to allow the U shaped bend 41 to reach entirely through the hollow 34 of the tube and extend out the opposite end when the headgear pad FIG. 3 is gathered slightly FIG. 6 along the length of the installation tool FIG. 4. The installation tool FIG. 4. has at the free end of each leg a curl 42 to protect the user from sharp points, to allow a gripping area for the fingers and as stoppage points for the insertion tool into the headgear pad FIG. 3. The width 43 containing both curls 42 of the installation tool FIG. 4 is slightly larger than the inside diameter of the hollow 34 area of the tube to prevent complete insertion of the tool FIG. 4.

FIG. 5 shows the installed headgear pad FIG. 3 and one appropriate location for its use with the soft outer layer 30 of the material 29 resting between the skin of the wearer and the strap bearing the pressure of the interface. The attachments 32 extend outward from the face allowing ease of access for the adjustment of the pad along the straps 21, 22. The hollow 34 of the headgear pad FIG. 3 is filled with the headgear strap 21 bulk. This being only one location that the headgear pad FIG. 3 could be located on the headgear; it could also be located on the upper strap 22 protecting the portion of the wearer's head above the ears, or additional headgear pads FIG. 3 could be located on both straps 21, 22 at one time.

FIGS. 6 and 7 show the installation steps of the headgear pad FIG. 3 onto the strap 21 of the headgear 20. After removing the interface from the headgear 20, the headgear pad FIG. 3 can be installed. In FIG. 6, the installation tool FIG. 4 U shaped bend 41 is inserted completely through the compressed headgear pad FIG. 3 and extends out the other end of the hollow 34. The headgear strap 21 is threaded through the U shaped bend 41 and secured with the strap tab 23 onto the installation tool FIG. 4. FIG. 7 shows the effect after force is applied to the curled ends 42 on the insertion tool FIG. 4 and the attachments 32 to urge the headgear pad FIG. 3 over the strap 21. After the headgear pad FIG. 3 is on the strap 21, the wire tool FIG. 4 is removed from the strap 21 and the interface is reattached to the headgear 20. Adjustment by way of the attachments 32 can position the strap pad FIG. 3 in a place most comfortable.

FIG. 8 shows the construction method of the preferred embodiment of the invention. A rectangular piece of material 29 is shaped into a hollow tube FIG. 3. The finished outward surface 30 of the material 29 is folded so the long sides are touching at the edges. The attachments 32 are sandwiched with their cut ends extending to the edge of the long side of the fabric inside the folded material 29. A reinforced seam 35 is sewn attaching both sides of the material 29 as well as the attachments 32 securing the material 29 into a hollow tube. This tube is further inverted so that the outer surface 30 is exposed and the attachments 32 extend away from the headgear pad FIG. 3. and the seam allowances 31 extend into the hollow 34.

FIG. 9 shows the construction of an alternative embodiment with 2 layers of rectangular shaped material 37 and 38 and no attachments 32. The inner layer of fabric 37 is a fabric of sufficient pile to provide cushion to human skin and a one directional stretch. The inner layer of fabric 37 is smaller in width than the outer layer of fabric 38 by an amount to allow for the inner layer 37 to lay flat against the inside of the tube formed by the outer layer of fabric 38 after the tube is sewn with seam 39 and inverted so that the seam allowances 31 extend into the hollow of the tube 34. The construction technique entails folding the material, bringing the long sides of both layers 37 and 38 to touch at the edge and seaming through all 4 layers with a reinforced seam of thread 39 holding the tube securely.

FIG. 10 shows an alternative embodiment of the invention using two layers, although more layers could be used. The outermost layer of fabric 38 can be decorative only and not necessarily of sufficient pile to provide padding.

FIG. 11 shows an alternate embodiment installed on an example of a nasal pillows headgear 24. No tool is necessary because of the rigid structure of the headgear strap 26 and it will slide easily into the alternate embodiment of the headgear pad FIG. 10. Alternative embodiments and methods would become apparent after considering this headgear pad FIG. 3 and are not included.

1. A pad of the type comprising a supple body of a material having pile depth sufficient to provide protection to the skin of a human, the improvement wherein said pad has a shape, style and size to improve a headgear strap:
a. a padding member comprising at least one layer of material,
b. dimension of approximate size to surround said headgear strap in close proximity and extending a predetermined length along the strap,
d. a seam of thread joining said padding member at the sides whereby a human wearing said headgear comprising a facial device and straps can do so in comfort without lasting physical impressions.

2. The padding of claim 1 wherein said material comprises fleece, micro fleece, cotton, silk, polyester, flannel, micro suede, and others of similar textures and weights whereby a thickness can be maintained between the headgear strap and the skin of the wearer.

3. The padding of claim 1 wherein said decorative material may be colorful and of such design whereby wearer can express personal style.

4. The padding of claim 1 wherein said material is sufficiently supple to be folded against itself to form the shape of a hollow tube,
a. of dimension capable of surrounding headgear strap in close proximity.

6. The padding in claim 1 whereas said decorative material are held together in close proximity forming a tube by a thread seam, with seam allowances protruding into said tube for the entire length of the tube.

7. A padding comprising a body of more than one layer of a supple material comprising at least one material having pile depth sufficient to provide cushion to the skin of a human and a decorative material, the improvement wherein said pad has a shape, style and size to improve a headgear strap.

8. The padding of claim 7 wherein said decorative material is substantially wider on its shortest side to surround the inner layer of said one material having pile depth sufficient to provide cushion to the skin of a human.

9. The padding of claim 7 wherein the layers of said supple material are secured together to form a hollow tube by a thread seam.

10. The padding of claim 7 wherein the inner layer of supple material lays flat against the inside of the tube

5. The padding of claim 1 wherein said attachments comprise satin ribbon, cotton ribbon, fabric strips and any similar narrow materials,
a. said narrow material of claim 5 is of predetermined length

11. An installation device comprised of a rigid member of predetermined length and strength comprising wire, wood, plastic, or other semi-rigid, bendable material.

12. The installation device of claim 11 formed into a bend forming parallel lengths sufficiently narrow to be placed into the hollow center of the padding tube.

13. The installation device of claim 11 terminating in curved forms of sufficient size and width apart to restrain said installation device from completely entering the hollow tube.

14. The installation device of claim 11 wherein said curved form provides a gripping surface for providing force to urge the headgear strap through the hollow tube of the pad.

15. The installation device of claim 11 wherein said curved form encloses the terminating end shielded from users skin.
